(19) United States
(12) Patent Application Publication
(10) Pub. No.: US 2021/0121044 A1
(43) Pub. Date: Apr. 29, 2021

(54) ENDOSCOPE SYSTEM

(71) Applicant: OLYMPUS CORPORATION, Tokyo (JP)

(72) Inventor: Masahide YAMAKI, Hachioji-shi (JP)

(73) Assignee: OLYMPUS CORPORATION, Tokyo (JP)

(21) Appl. No.: 17/139,187

(22) Filed: Dec. 31, 2020

Related U.S. Application Data
(63) Continuation of application No. PCT/JP2019/026605, filed on Jul. 4, 2019.

(30) Foreign Application Priority Data
Jul. 4, 2018 (JP) 2018-127592

(51) Int. Cl. A61B 1/00

(52) U.S. Cl. CPC A61B 1/00006; A61B 1/00016; A61B 1/00121

(57) ABSTRACT

An endoscope system includes a medical device that is capable of communicating with a peripheral medical device, a control terminal apparatus that generates a control command for controlling the medical device, an adapter that communicably connects the control terminal apparatus and the medical device and is capable of being communicably connected to a plurality of network apparatuses over a network, and a second monitoring apparatus that monitors a state of connection between the medical device and the peripheral medical device and a state of connection between the adapter and the network apparatuses established over the network.

### CROSS-REFERENCE TO RELATED APPLICATION

This application is based upon and claims the benefit of priority from prior Japanese Patent Application No. 2018-127592, filed Jul. 4, 2018, the entire contents of which are incorporated herein by this reference.

This application is a continuation application of International Application PCT/JP2019/026605 filed on Jul. 4, 2019, the entire contents of which are incorporated herein by reference.

### FIELD

The present invention relates to an endoscope system used in the medical field.

### BACKGROUND

Various proposals for improving the operability or the like of apparatuses and systems used in the medical field have conventionally been offered.

For example, Japanese Laid-open Patent Publication No. 2014-188082 proposes a portable console terminal that allows a plurality of modalities of different types to be operated and simplifies the tasks of updating console application programs. Japanese Laid-open Patent Publication No. 2016-96941 proposes an endoscope apparatus that allows optimum functions corresponding to the type of an endoscope apparatus to be easily added. Japanese Laid-open Patent Publication No. 2008-200173 proposes an electronic endoscope system that allows programs operated in a processor for an electronic endoscope or in the electronic endoscope or parameters operated by the programs to be customized at a user request.

### SUMMARY

In an aspect of the present invention, an endoscope system includes a medical device that is used to observe or treat a subject and is capable of communicating with a peripheral medical device, a control terminal apparatus that generates a control command for controlling the medical device, an adapter that communicably connects the control terminal apparatus and the medical device and is capable of being communicably connected to a plurality of network apparatuses over a network, and a second monitoring apparatus that monitors a state of connection between the medical device and the peripheral medical device and a state of connection between the adapter and the network apparatus established over the network.

### BRIEF DESCRIPTION OF DRAWINGS

FIG. 1 illustrates a configuration example of an endoscope system in accordance with an embodiment;

FIG. 2 specifically illustrates a configuration example of a portion of an endoscope system in accordance with an embodiment;

FIG. 3 illustrates a hardware configuration example of first and second monitoring apparatuses;

FIG. 4 is a flowchart illustrating an example of a flow from delivery of a portable terminal to processes such as information transmission to a server after execution of processing in an endoscope system; and

FIG. 5 illustrates a configuration example of an endoscope system in accordance with a variation.

### DESCRIPTION OF EXEMPLARY EMBODIMENTS

In recent years, there has been need to improve inspection efficiency by customizing endoscope systems formed from video processors, monitors, and medical devices such as electric knife apparatuses through addition of new software or hardware thereto in consideration of how such systems are used in facilities.

However, when, for example, a consumer application is newly installed for the purpose of improving inspection efficiency, the newly installed application could affect operations of already installed medical applications.

Accordingly, embodiments disclosed in the following provide an endoscope system capable of monitoring the influence of a newly installed application on operations of already installed medical applications.

The following describes embodiments of the present invention by referring to the drawings.

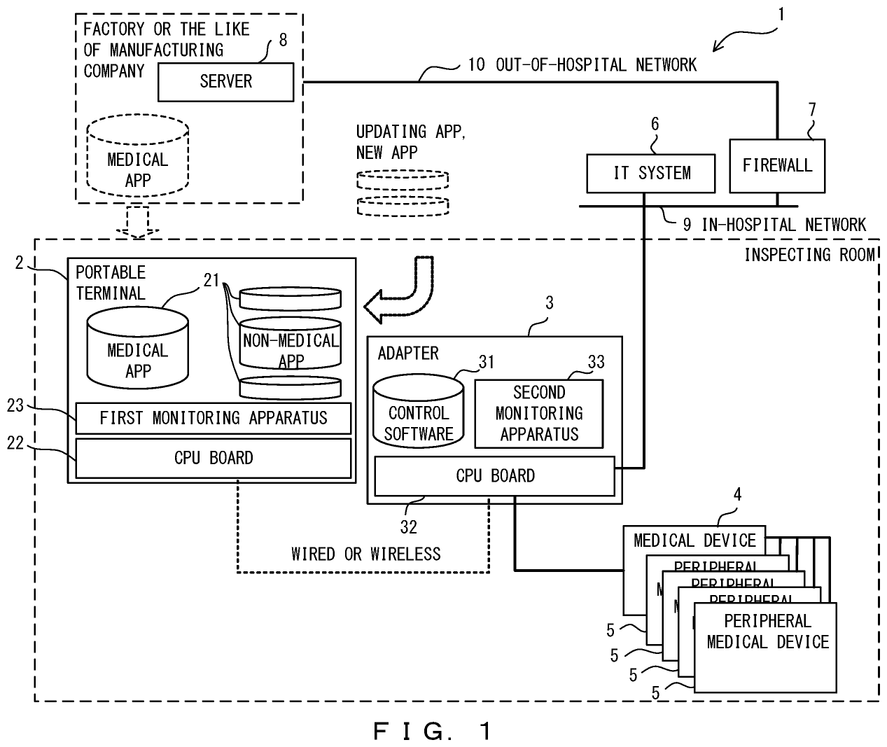

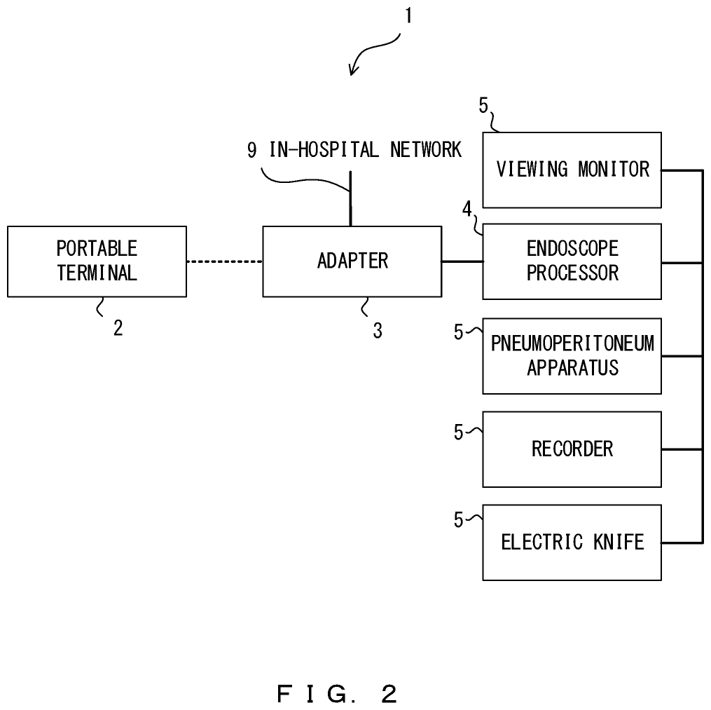

FIG. 1 illustrates a configuration example of an endoscope system in accordance with an embodiment. FIG. 2 specifically illustrates a configuration example of a portion of an endoscope system in accordance with an embodiment.

As depicted in FIG. 1, an endoscope system 1 includes a portable information terminal apparatus (hereinafter, “portable terminal”) 2, an adapter 3, a medical device 4, a plurality of peripheral medical devices 5, an information technology (IT) system 6, a firewall 7, and a server 8.

The portable terminal 2, the adapter 3, the medical device 4, and the plurality of peripheral medical devices 5 are used in an inspecting room in a hospital. The IT system 6 and the firewall 7 are provided within the hospital. The server 8 is provided within a factory or the like of a manufacturing company for the portable terminal 2.

The manufacturing company for the portable terminal 2 develops medical applications (hereinafter, “medical apps”) to be installed in the portable terminal 2 and applications for updating the medical apps (hereafter, “updating apps”). The medical apps are applications (hereinafter, “apps”) for allowing the medical device 4 and the plurality of peripheral medical devices 5 to be controlled, and in a development stage therefor, operations are ensured for expected use conditions. The expected use conditions include, for example, a condition in which the medical apps are installed in the portable terminal 2 and the portable terminal 2 is used in an expected system configuration. The medical device 4 and the peripheral medical devices 5 are included in the expected system configuration.

The portable terminal 2 and the adapter 3 are communicably connected, the adapter 3 and the medical device 4 are communicably connected, and the medical device 4 and each of the peripheral medical devices 5 are communicably connected. For example, the portable terminal 2 may be configured to be capable of being attached to and detached from to the adapter 3; when the portable terminal 2 is attached to the adapter 3, these two may be communicably connected in a wired manner; and when the portable terminal 2 is detached from the adapter 3, these two may be communicably connected in a wireless manner.

The adapter 3 and the IT system 6 are connected to an in-hospital network 9 and communicably connected to each other over the in-hospital network 9. The in-hospital network 9 is provided within the hospital and is, for example, a local area network (LAN). The in-hospital network 9 and an out-of-hospital network 10 are connected by the firewall 7. The out-of-hospital network 10 is provided outside the hospital and is, for example, the Internet. The server 8 is connected to the out-of-hospital network 10 and communicably connected to the adapter 3 and the like via the out-of-hospital network 10, the firewall 7, and the in-hospital network 9.

Apparatuses connected to the in-hospital network 9 and those connected to the out-of-hospital network 10 may be referred to as network apparatuses.

For example, the portable terminal 2 may be a tablet information terminal apparatus or a smartphone.

Installing a medical app in the portable terminal 2 may allow the portable terminal 2 to control the medical device 4 and the plurality of peripheral medical devices 5. In particular, the medical app may generate and transmit control commands for controlling the medical device 4 or the plurality of medical devices 5. For the transmission of the control commands, control commands for controlling the medical device 4 are transmitted to the medical device 4 via the adapter 3, and control commands for controlling the peripheral medical devices 5 are transmitted to the peripheral medical devices 5 via the adapter 3 and the medical device 4.

A non-medical app can be installed in the portable terminal 2 as a new app. An updating app for the medical app or an updating app for the non-medical app can be installed in the portable terminal 2. The non-medical app does not generate or transmit control commands for controlling medical devices such as the medical device 4 and the plurality of peripheral medical devices 5 and is, for example, a consumer app such as a printing app or a sound input assistance app.

The portable terminal 2 includes a hard disk drive (HDD) 21, a central-processing-unit (CPU) board 22, and a first monitoring apparatus 23.

The HDD 21 stores programs such as medical apps and non-medical apps installed in the portable terminal 2.

The CPU board 22 includes a memory storing an operating system (OS), a CPU for executing the OS or a program such as an app stored in the HDD 21, a communication interface for allowing for a communication with the adapter 3, and the like.

The first monitoring apparatus 23 monitors installation of a new app into the portable terminal 2. When the endoscope system 1 is activated or when a new app is installed in the portable terminal 2, the first monitoring apparatus 23 determines whether operations of medical apps on the portable terminal 2 satisfy a predetermined operation condition. When a determination result indicating that the predetermined operation condition is not satisfied is obtained, the first monitoring apparatus 23 transmits determination result information pertaining to the determination result to the server 8. The determination result information may include a monitoring log provided by the first monitoring apparatus 23. This monitoring log is also information indicating installation history for apps installed in the portable terminal 2.

Although not illustrated, the portable terminal 2 also includes an input apparatus such as a touch panel, an output apparatus such as a liquid crystal display, and the like.

The adapter 3 is an apparatus that communicably connects the portable terminal 2 and the medical device 4.

The adapter 3 includes an HDD 31, a CPU board 32, and a second monitoring apparatus 33.

The HDD 31 stores programs such as software for control (hereinafter, “control software”). For example, the control software may be software for controlling a communication performed via the adapter 3 or software for performing control for streaming-delivering video signals generated by the medical device 4 in real time to the portable terminal 2, the IT system 6, or the like.

The CPU board 32 may include a memory storing an OS, a CPU for executing the OS or a program such as control software stored in the HDD 31, a communication interface for allowing for a communication with the portable terminal 2, the medical device 4, the IT system 6, the server 8, and the like.

The second monitoring apparatus 33 monitors the state of connection between the medical device 4 and the peripheral medical devices 5 and the state of connection between the adapter 3 and the IT system 6 established over the in-hospital network 9. When the endoscope system 1 is activated or when a change is made to the state of connection, the second monitoring apparatus 33 determines whether the state of connection at that time satisfies a predetermined connection condition. When a determination result indicating that the predetermined connection condition is not satisfied is obtained, the second monitoring apparatus 33 transmits determination result information pertaining to the determination result to the server 8. The determination result information may include a monitoring log provided by the second monitoring apparatus 33.

The adapter 3 may also include an output apparatus such as a liquid crystal display, an input apparatus such as a touch panel, and the like.

The medical device 4 and the plurality of peripheral medical devices 5 are used to observe or treat a subject.

Specifically, the medical device 4 may be an endoscope video processor (hereinafter, “endoscope processor”) as depicted in FIG. 2 and generate a video signal on the basis of an imaging signal output from an imaging unit of an endoscope (not illustrated) inserted into a subject. The medical device 4 can be operated in accordance with a control command transmitted from the portable terminal 2.

As depicted in FIG. 2, the plurality of peripheral medical devices 5 may be a viewing monitor for displaying a video signal generated by the endoscope processor, i.e., the medical device 4, a recorder for recording a video signal generated by the endoscope processor, i.e., the medical device 4, a pneumoperitoneum apparatus for implementing pneumoperitoneum using carbon dioxide, and an electric knife apparatus for implementing incision or hemostasis by causing a high-frequency current to flow through a body site. Each of the peripheral medical devices 5 can also be operated in accordance with a control command transmitted from the portable terminal 2.

The IT system 6 may include a personal computer (PC) and a server and manage inside information of the hospital, including medical information.

The firewall 7 may interrupt communications determined to be illicit in accordance with a predetermined standard, among communications between the in-hospital network 9 and the out-of-hospital network 10.

The server 8 accumulates determination result information transmitted from the first monitoring apparatus 23 and determination result information transmitted from the second monitoring apparatus 33. For example, the accumulated determination result information may be used by the manufacturing company for the portable terminal 2, i.e., a company that develops medical apps, when verifying the operation and performance of medical apps.

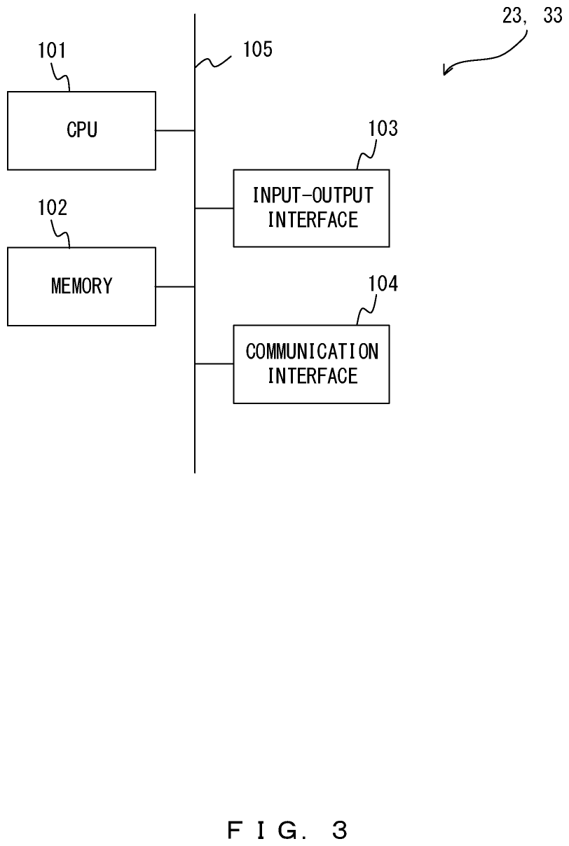

FIG. 3 illustrates a hardware configuration example of the first and second monitoring apparatuses.

As depicted in FIG. 3, the first monitoring apparatus 23 and the second monitoring apparatus 33 each include a CPU 101, a memory 102, an input-output interface 103, and a communication interface 104, which are connected to each other by a bus 105.

When the hardware configuration depicted in FIG. 3 is the first monitoring apparatus 23, the CPU 101 is an operational device that executes a program for processing performed by the first monitoring apparatus 23. The memory 102 is a random access memory (RAM) and a read only memory (ROM). The RAM is used as a work area or the like for the CPU 101. The ROM stores a program and information necessary for execution of the program in a nonvolatile manner. The input-output interface 103 is connected to the CPU board 22 and inputs/receives signals to/from the CPU board 22. The communication interface 104 is an interface for allowing for a communication with the adapter 3. Note that the CPU board 22 may be provided with the hardware configuration depicted in FIG. 3 as the first monitoring apparatus 23.

When the hardware configuration depicted in FIG. 3 is the second monitoring apparatus 33, the CPU 101 is an operational device that executes a program for processing performed by the second monitoring apparatus 33. For the memory 102, the above descriptions should be referred to. The input-output interface 103 is connected to the CPU board 32 and inputs/receives signals to/from the CPU board 32. The communication interface 104 is an interface for allowing for communications with the portable terminal 2, the medical device 4, the IT system 6, the server 8, and the like. Note that the CPU board 32 may be provided with the hardware configuration depicted in FIG. 3 as the second monitoring apparatus 33.

The first monitoring apparatus 23 and the second monitoring apparatus 33 are not limited to the hardware configuration depicted in FIG. 3 and may each be implemented by a circuit, e.g., a field-programmable gate array (FPGA) or an application specific integrated circuit (ASIC). Also in this case, the CPU board 22 may be provided with a circuit that functions as the first monitoring apparatus 23, and the CPU board 32 may be provided with a circuit that functions as the second monitoring apparatus 33.

The following describes processing performed in the endoscope system 1.

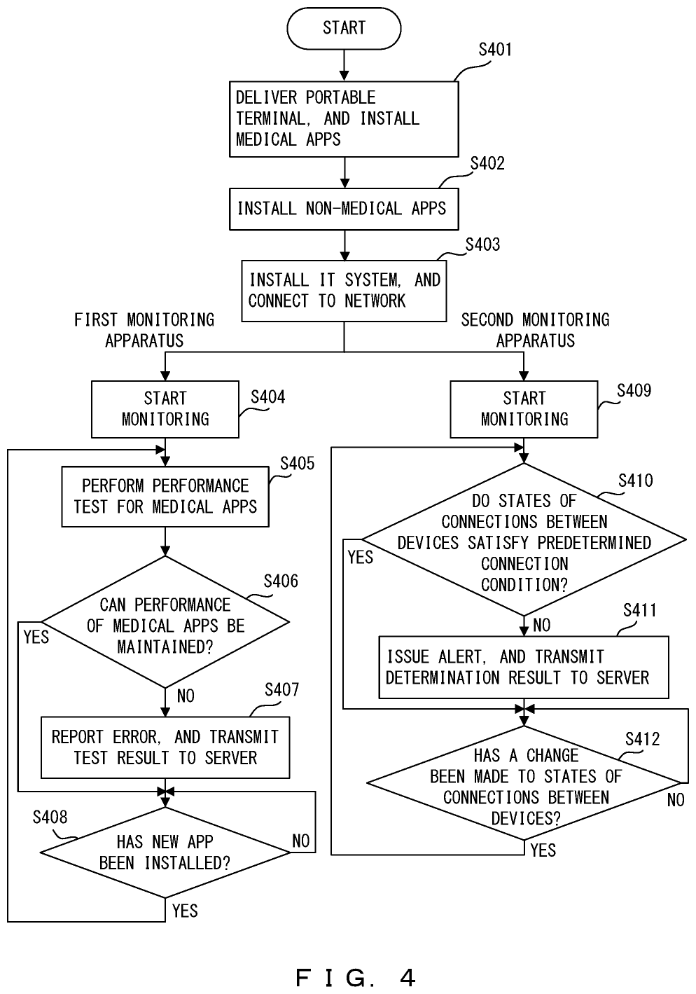

FIG. 4 is a flowchart illustrating an example of a flow from delivery of the portable terminal to processes such as information transmission to the server after execution of processing in the endoscope system.

As depicted in FIG. 4, this flowchart is such that first, the portable terminal 2 and medical apps are delivered to the hospital, and the medical apps are installed in the portable terminal 2 (S401). Next, non-medical apps are installed in the portable terminal 2 in accordance with the user's preference or in consideration of facilities in an inspecting room in which the portable terminal 2 is to be used (S402). Afterward, the IT system 6 is installed, and a connection to the in-hospital network 9 is established (S403), thereby establishing the endoscope system 1 depicted in FIG. 1.

When the endoscope system 1 is activated, the first monitoring apparatus 23 performs the processes of S404-S408, and the second monitoring apparatus 33 performs the processes of S409-S412.

In the processes of S404-S408, the first monitoring apparatus 23 first starts to monitor installation of a new app into the portable terminal 2 (S404).

Next, the first monitoring apparatus 23 performs a performance test for the medical apps installed in the portable terminal 2 (S405) and determines whether the test result indicates that the performance of the medical apps can be maintained (S406). In the performance test for the medical apps, the medical apps are operated, and it is determined whether the operation result satisfies a predetermined operation standard. It can be determined that the performance of the medical apps can be maintained when the predetermined operation standard is satisfied.

When the determination result from S406 is NO, the first monitoring apparatus 23 gives an error report and transmits information pertaining to the test result to the server 8 (S407). For example, the error report may be given by causing the portable terminal 2 to display an error message that the performance of the medical apps cannot be maintained. The information pertaining to the test result that is transmitted to the server 8 may include a monitoring log provided by the first monitoring apparatus 23.

When the determination result from S406 is YES or after the process of S407 is performed, the first monitoring apparatus 23 determines whether a new app has been installed in the portable terminal 2 (S408). When the determination result from S408 is Yes, the process returns to S405, and when the determination result from S408 is NO, the determination is performed again.

According to the processes performed by the first monitoring apparatus 23 (S404-S408), when the endoscope system 1 is activated or when a new app is installed in the portable terminal 2, the performance test for medical apps is performed. When the test result indicates that the performance of the medical apps cannot be maintained, information pertaining to the test result is transmitted to the server 8 together with an error report.

In the processes of S409-S412, the second monitoring apparatus 33 first starts to monitor the state of connection between the medical device 4 and the peripheral medical devices 5 and the state of connection between the adapter 3 and the IT system 6 established over the in-hospital network 9 (S409). For example, this monitoring may be implemented by the second monitoring apparatus 33 periodically communicating with the medical device 4, the peripheral medical devices 5, and the IT system 6 so as to obtain the device identifier (ID) of the medical device 4, the device IDs of the peripheral medical devices 5, and the Internet Protocol (IP) addresses or network interface card (NIC) IDs of the network apparatuses included in the IT system 6.

Next, the second monitoring apparatus 33 determines whether the state of connection between the medical device 4 and the peripheral medical devices 5 and the state of connection between the adapter 3 and the IT system 6 established over the in-hospital network 9 satisfy a predetermined connection condition (S410). The predetermined connection condition is a device connection configuration expected in the development stage for the medical apps. In a case where the devices are connected in accordance with the device connection configuration, at least when only the medical apps have been installed in the portable terminal 2, the operations of the medical apps are ensured.

When the determination result from S410 is NO, the second monitoring apparatus 33 issues an alert and transmits information pertaining to the determination result to the server 8 (S411). For example, the alert may be issued by communicating with and causing the portable terminal 2 to display an alert message that an unexpected device connection has been established. The information pertaining to the determination result that is transmitted to the server 8 may include information pertaining to the states of device connections at that time and also include a monitoring log provided by the second monitoring apparatus 33.

When the determination result from S410 is YES or after the process of S411 is performed, the second monitoring apparatus 33 determines whether a change has been made to the states of connections being monitored (S412). For example, a change having been made to the states of connections may mean that the number of peripheral medical devices 5 connected to the medical device 4 has increased or decreased, that a peripheral medical device 5 connected to the medical device 4 has been replaced, that the number of network apparatuses included in the IT system 6 has increased or decreased, or that the configuration of the IT system 6 has been changed. When the determination result from S412 is Yes, the process returns to S410, and when the determination result from S412 is NO, the determination is performed again.

According to the processes to be performed by the second monitoring apparatus 33 (S409-S412), when the endoscope system 1 is activated or when a change is made to the state of connection being monitored, it is determined whether the states of device connections satisfy the predetermined connection condition. When the determination result indicates that the predetermined connection condition is not satisfied, information pertaining to the determination result is transmitted to the server 8 together with an alert.

In embodiments, as described above, every time a new app is installed in the portable terminal 2, the performance test therefor is performed so that the influence of the newly installed app on operations of medical apps can be monitored.

In addition, when the result of the performance test for a medical app indicates that the performance of the medical app cannot be maintained, information pertaining to the result is transmitted to the server 8, and when the states of device connections do not satisfy a predetermined connection condition, information pertaining to the connection condition at that time is transmitted to the server 8. Thus, the manufacturing company for the portable terminal 2, i.e., a company that develops medical apps, can verify the operations and performances of the medical apps on the basis of such information.

There are various variations of the embodiments.

For example, the second monitoring apparatus 33 may further monitor the device setting state of the medical device 4 and the device setting states of the peripheral medical devices 5 connected to the medical device 4. For example, this monitoring may be implemented by the second monitoring apparatus 33 periodically communicating with the medical device 4 and the peripheral medical devices 5 so as to obtain information pertaining to the device settings thereof.

For example, the first monitoring apparatus 23 may further periodically transmit monitoring logs obtained by this apparatus to the server 8, and the second monitoring apparatus 33 may further periodically transmit monitoring logs obtained by this apparatus to the server 8. The server 8 may further accumulate the monitoring logs.

In S407 depicted in FIG. 4, for example, the first monitoring apparatus 23 may incorporate a monitoring log provided by the second monitoring apparatus 33 into information pertaining to a test result to be transmitted to the server 8. In this case, the monitoring log provided by the second monitoring apparatus 33 is acquired from the second monitoring apparatus 33 through, for example, a communication with the second monitoring apparatus 33.

In S411 depicted in FIG. 4, for example, the second monitoring apparatus 33 may incorporate a monitoring log provided by the first monitoring apparatus 23 into information pertaining to a determination result to be transmitted to the server 8. In this case, the monitoring log provided by the first monitoring apparatus 23 is acquired from the first monitoring apparatus 23 through, for example, a communication with the first monitoring apparatus 23.

For example, the endoscope system 1 may not include the adapter 3.

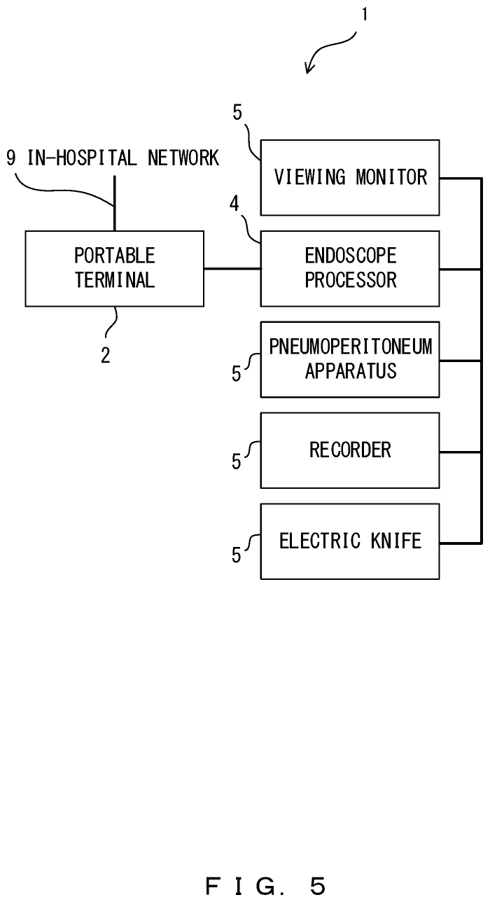

FIG. 5 illustrates a configuration example of an endoscope system in accordance with a variation without the adapter. FIG. 5 corresponds to FIG. 2, and indications of the IT system 6, the firewall 7, the out-of-hospital network 10, and the server 8 are omitted therein.

As depicted in FIG. 5, the endoscope system 1 in accordance with the variation is such that the portable terminal 2 is communicably connected directly to an endoscope processor consisting of the medical device 4. The portable terminal 2 is also connected to the in-hospital network 9. As a result, the portable terminal 2 is communicably connected to the IT system 6 over the in-hospital network 9 and communicably connected to the server 8 via the in-hospital network 9, the firewall 7, and the out-of-hospital network 10.

The endoscope system 1 in accordance with this variation is such that the HDD 21 of the portable terminal 2 stores the program such as control software that would be stored in the HDD 31 of the adapter 3 in the above-described embodiments, and the portable terminal 2 is provided with the second monitoring apparatus 33 that would be provided in the adapter 3 in the above-described embodiments. In this case, the second monitoring apparatus 33 monitors the state of connection between the portable terminal 2 and the IT system. 6, rather than the state of connection between the adapter 3 and the IT system 6.

The endoscope system 1 in accordance with this variation can have a simplified configuration.

In embodiments, for example, a company other than the manufacturing company for the portable terminal 2 may develop medical apps and updating apps therefor. Thus, a company that develops medical apps and updating apps therefor may be different from the manufacturing company for the portable terminal 2. In this case, the server 8 may be provided for the company that develops medical apps and updating apps therefor.

Alternatively, for example, a plurality of companies may develop medical apps and updating apps therefor. In this case, medical apps and updating apps therefor may be developed by the manufacturing company for the portable terminal 2 and one or more companies other than the manufacturing company for the portable terminal 2 or only by a plurality of companies other than the manufacturing company for the portable terminal 2. In this case, the server 8 may be provided for at least one of the companies that develop medical apps and updating apps therefor.

In embodiments, for example, other medical apps may further be installed in the portable terminal 2. The other medical apps are apps for allowing medical devices other than the medical device 4 and the plurality of medical devices 5 to be controlled, e.g., an app for allowing a medical lighting device such as a shadowless lamp to be controlled, an app for allowing a medical bed to be controlled. The other medical apps may be developed by the manufacturing company for the portable terminal 2 or may be developed by a company other than the manufacturing company for the portable terminal 2.

In embodiments, for example, a company that develops medical apps and updating apps therefor may further develop non-medical apps and updating apps therefor. For example, non-medical apps in this case may be apps such as a patient identifier (ID) assistance app for managing patient information, an image management app for managing medical images, and a reporting app for assisting in creation of a report.

In embodiments, for example, when a new app is installed into the portable terminal 2, the portable terminal 2 may transmit information pertaining to a list of already installed apps to the server 8. This is performed to, for example, analyze the influences of combinations of apps or to search customer needs.

In embodiments, for example, when a new app is installed into the portable terminal 2, the portable terminal 2 may transmit the version of the OS stored in the memory of the CPU board 22 of the portable terminal 2 to the server 8. This is performed to, for example, analyze an influence on the app that could be exerted by the version of the OS.

In exemplary embodiments, for example, the portable terminal 2 may transmit, to the server 8, data (which may include apps) held therein before installation of a new app. In particular, the data may be stored in the server 8 as backup. This is performed to, for example, allow operations of the portable terminal 2 to be restored if the operations fail due to the influence of installation of the new app.

In exemplary embodiments, for example, the portable terminal 2 may be provided with separate memories (RAMS) for use in execution of a medical app or for use in execution of a non-medical app. This is intended to, for example, reduce influences on the medical device 4 and the plurality of medical devices 5 that could be exerted if a memory (RAM) is excessively loaded.

The present invention is not simply limited to the embodiments described herein. Components of the embodiments may be embodied in a varied manner in an implementation phase without departing from the gist of the invention. A plurality of components disclosed with reference to the described embodiments may be combined, as appropriate, to achieve various inventions. For example, some of the components indicated with reference to embodiments may be omitted. In addition, components from different embodiments may be combined as appropriate.

What is claimed is:

1. An endoscope system comprising:
a medical device configured to observe or treat a subject and configured to communicate with a peripheral medical device;
a control terminal apparatus, including a processor and a memory, that generates a control command for controlling the medical device;
an adapter configured to connect, either based on a wired connection or wirelessly, to the control terminal apparatus and the medical device and is configured to communicate with a plurality of network apparatuses over a network; and
a second monitoring apparatus configured to monitor a connection state between the medical device and the peripheral medical device and a connection state between the adapter and the network apparatuses established over the network.

2. The endoscope system of claim 1, wherein
when the endoscope system is activated or when a change is made to the connection state, the second monitoring apparatus determines whether the connection state at that time satisfies a predetermined connection condition, and when a determination result indicating that the predetermined connection condition is not satisfied is obtained, the second monitoring apparatus transmits determination result information pertaining to the determination result to an external server.

3. The endoscope system of claim 1, wherein
the adapter includes the second monitoring apparatus.

4. The endoscope system of claim 1, wherein
the control terminal apparatus includes the second monitoring apparatus.

5. The endoscope system of claim 1, wherein
the control terminal apparatus and the adapter are connected in a wireless manner.

6. The endoscope system of claim 1, wherein
the control terminal apparatus has a first non-transitory computer readable application for generating a control command for controlling the medical device installed therein and is capable of having a second non-transitory computer readable application newly installed therein, and
the endoscope system further comprises a first monitoring apparatus configured to monitor installation of the second non-transitory computer readable application into the control terminal apparatus.

7. The endoscope system of claim 6, wherein
when the endoscope system is activated or when the second non-transitory computer readable application is installed in the control terminal apparatus, the first monitoring apparatus determines whether an operation of the first non-transitory computer readable application on the control terminal apparatus satisfies a predetermined operation condition, and when a determination result indicating that the predetermined operation condition is not satisfied is obtained, the first monitoring apparatus transmits determination result information pertaining to the determination result to an external server.

8. The endoscope system of claim 6, wherein
the first monitoring apparatus is configured to transmit history information indicating installation history for the second application to the external server.

9. The endoscope system of claim 6, wherein
the control terminal apparatus includes the first monitoring apparatus.

10. The endoscope system of claim 6, wherein
a non-transitory computer readable application that does not generate a control command for controlling the medical device can be installed in the control terminal apparatus as the second non-transitory computer readable application.
